(19) United States
(12) Patent Application Publication
(10) Pub. No.: US 2025/0303121 A1
(43) Pub. Date: Oct. 2, 2025

(54) VARIABLE DIAMETER INFLATABLE MEDICAL BALLOON AND RELATED METHODS

(71) Applicant: BARD PERIPHERAL VASCULAR, INC., Franklin Lakes, NJ (US)

(72) Inventors: Nicolas Coracides, Tempe, AZ (US); David Tze, Phoenix, AZ (US)

(21) Appl. No.: 18/873,030

(22) PCT Filed: Jul. 29, 2022
(86) PCT No.: PCT/US2022/038872

§ 371 Date: Dec. 9, 2024

(51) Int. Cl. A61M 25/10

(52) U.S. Cl. CPC A61M 25/10; A61M 2025/1056; A61M 2025/1068; A61M 2025/1075; A61M 2025/1084

(57) ABSTRACT

A medical apparatus (10) includes an inflatable medical balloon (12) formed of a compliant material having one or more fibers (14) embedded therein, which may be braided or form intersections having a first lower angle at a first lower state of inflation of the medical balloon and a second higher at a second higher state of inflation of the medical balloon. A catheter shaft may connect to a tubing for forming the inflatable medical balloon. The outer diameter of the tubing may be substantially flush with outer diameters of the opposed ends of the catheter shaft. A guidewire tube may pass through the catheter shaft and the tubing and connect to a distal tip of the catheter shaft. A proximal hub includes a first port for supplying an inflation fluid to the catheter shaft and a second port adapted for receiving a guidewire for passing through the catheter shaft.

### BACKGROUND

Medical balloons are often used to open or expand open body spaces restricted by tough tissues such as strictures, scarring or calcified areas. In these applications, medical balloons having high operating and burst pressures may be required. For example, dilation balloons are used in angioplasty, a procedure in which the balloon may be used to expand a stenoic lesion. In these applications it is desirable to make the outer wall of the dilation balloon as thin as possible while still maintaining the required pressure rating or burst strength. It is also desirable that the balloon exhibit a high degree of puncture resistance.

Medical balloons are generally referred to as compliant, non-compliant and semi-compliant. Balloon compliance is a term used to describe the change in a balloon's diameter as a function of pressure. Low pressure compliant medical balloons are typically formed from elastomers such as latex, polyurethane and other thermoplastic elastomers. Low pressure compliant medical balloons may expand by 100% or greater upon inflation. Compliant medical balloons are typically used for fixation and occlusion.

Alternatively, high pressure non-compliant dilation balloons expand very little, if at all, when pressurized from a nominal diameter to a rated burst pressure. The rated burst pressure is the maximum pressure at which there is a statistical 95% confidence level that 99.9% of the population of balloons will not burst. High pressure non-compliant balloons may have rated burst pressures of up to 20 atmospheres or higher. Generally, high pressure, non-compliant balloons are formed from relatively inelastic materials such as oriented highly crystalline polyethylene terephthalate (PET) films. Such PET films provide high tensile strength, and may be used to form balloons with thin walls having high burst pressures. However, balloons formed from PET and similar materials having a high strength relative to wall thickness tend to be more susceptible to puncture. Balloons formed from PET also tend to be stiffer than balloons made from other more compliant materials. The stiffness of the deflated balloon directly affects its “trackability,” i.e., its ability to traverse sharp turns or branches of the vessels or body cavities through which the balloon must pass. Balloons having more flexible walls generally provide better trackability.

In some applications, a compliant balloon may be more desirable than a non-compliant balloon. Compliant balloons tend to be less stiff than non-compliant balloons, resulting in better trackability. Compliant balloons may also provide better puncture resistance than non-compliant balloons. Thus, a practitioner may prefer a compliant balloon over a non-compliant balloon in procedures where there is a need for a balloon to expand to varying diameters, where the balloon must be threaded through small diameter blood vessels, and/or where the balloon has to traverse a torturous path. In some instances, a compliant dilation balloon may be used to pre-dilate a stenosis before stent placement. A practitioner may also prefer a compliant dilation balloon over a non-compliant balloon for stent placement and/or for post-stent dilation.

In order to reduce the profile of the balloon, dilation balloons may be formed with pleated walls. When the balloon is deflated (i.e., before or after inflation), these pleats are folded over, wrapped and/or rolled around the long axis of the balloon. Consequently, the thinner the wall material of the balloon, the smaller the diameter of the balloon-catheter assembly. A smaller diameter may be used with a smaller introducer, reducing patient discomfort. A smaller diameter also facilitates passage of the deflated balloon through narrow vessels, lumens or cavities of the body prior to deployment.

Accordingly, there exists a need for an inflatable medical balloon that can achieve the benefits of providing a variable diameter for dilation purposes, while having a high level of compliance, puncture resistance, high burst pressures and thin walls.

### SUMMARY

An object of the disclosure is to provide a variable diameter, inflatable medical balloon (which means the medical balloon has different diameters at different inflation pressures). The medical balloon may comprise a compliant tubing with fiber embedded in the wall of the tubing. The fiber may be in a braided pattern with either a consistent low winding angle or a variable winding angle along the length of the tubing. The proximal and distal end of the compliant tubing forming the medical balloon may be welded directly to opposed ends of a catheter shaft, which ends may have the same outer diameter as the tubing and thus be flush therewith in an uninflated condition to eliminate variability and otherwise reduce the outer profile of the resulting apparatus. The medical balloon may be inflated via a port associated with a proximal hub connected to the catheter shaft. In a deflated state or not under pressure, the compliant tubing may thus be flush with the catheter shaft. As the compliant tubing is inflated, the embedded fibers only allow for radial growth of the compliant material forming the medical balloon with increasing pressure and in a controlled manner as the fibers resist expansion.

In accordance with one aspect of the disclosure, a medical apparatus is provided. The medical apparatus includes an inflatable medical balloon of a variable diameter comprising an ultra-compliant material having one or more fibers embedded therein.

In one embodiment, the ultra-compliant material is selected from the group comprising silicone, polyurethane, hydrogel, or any polymer with extensibility over 30% and Young's modulus less than 100 MPa. In this or other embodiments, the one or more fibers comprise one or more braided fibers, and/or the one or more fibers comprise one or more fiber intersections having a first lower angle at a first lower state of inflation of the medical balloon and a second higher at a second higher state of inflation of the medical balloon.

The apparatus may further include a catheter shaft having opposed ends connected to a tubing forming the inflatable medical balloon. An outer diameter of the tubing may be substantially flush with outer diameters of the opposed ends of the catheter shaft in an uninflated condition. A guidewire tube may be provided within the catheter shaft and the tubing forming the inflatable medical balloon, the guidewire tube connected to a distal tip of the catheter.

A hub at a proximal end of the catheter shaft may include a first port for supplying an inflation fluid to the catheter shaft for expanding the tubing. The hub may further include a second port adapted for receiving a guidewire for passing through the catheter shaft.

According to a further embodiment, a medical apparatus includes a catheter shaft including opposed ends connected to a tubing. The tubing is formed of a compliant material with one or more fibers therein. Thus, the tubing forms an inflatable medical balloon of a variable diameter when the catheter shaft is pressurized as a result of the compliance of the compliant material.

In one embodiment, the compliant material comprises an ultra-compliant material. For example, the ultra-compliant material may be selected from the group comprising silicone, polyurethane, hydrogel, or any polymer with extensibility over 30% and Young's modulus less than 100 MPa.

The one or more fibers may comprise one or more braided fibers. The one or more fibers may comprise one or more fiber intersections having a first lower angle at a first lower state of inflation of the medical balloon and a second higher at a second higher state of inflation of the medical balloon.

An outer diameter of the tubing may be substantially flush with outer diameters of the opposed ends of the catheter shaft in an uninflated condition. A guidewire tube may be provided within the catheter shaft and the tubing forming the inflatable medical balloon, the guidewire tube connected to a distal tip of the catheter shaft.

A hub may be provided at a proximal end of the catheter shaft including a first port for supplying an inflation fluid to the catheter shaft for expanding the tubing. The hub may further include a second port adapted for receiving a guidewire for passing through the catheter shaft.

Still a further aspect of the disclosure pertains to a medical apparatus, comprising a catheter shaft connected to an inflatable medical balloon. The catheter shaft includes an outer surface flush with an outer surface of the inflatable medical balloon in an uninflated condition.

In one embodiment, the medical balloon comprises a tubing formed of a compliant material with one or more fibers therein. The compliant material may comprise an ultra-compliant material, such as one selected from the group comprising silicone, polyurethane, hydrogel, or any polymer with extensibility over 30% and Young's modulus less than 100 MPa.

The one or more fibers may comprise one or more braided fibers. The one or more fibers may comprise one or more fiber intersections having a first lower angle at a first lower state of inflation of the medical balloon and a second higher at a second higher state of inflation of the medical balloon.

The medical balloon may be attached to opposed ends of the catheter shaft. A guidewire tube may be provided within the catheter shaft and the inflatable medical balloon, the guidewire tube connected to a distal tip of the catheter shaft.

A hub may be provided at a proximal end of the catheter shaft including a first port for supplying an inflation fluid to the catheter shaft for expanding the medical balloon. The hub may further include a second port adapted for receiving a guidewire for passing through the catheter shaft.

### BRIEF DESCRIPTION OF THE DRAWINGS

The above and further advantages of the disclosure may be better understood by referring to the following description in conjunction with the accompanying drawings in which:

FIG. 1 illustrates a side view of catheter including a variable diameter medical balloon;

FIG. 1A is a cross-section taken along line 1A-1A of FIG. 1;

FIG. 2 is a side view of the catheter including the medical balloon in a first expanded state;

FIG. 3 is a side view of the catheter including the medical balloon in a second expanded state;

FIG. 4 is a cutaway side view of a portion of a medical balloon showing helical fibers having a low angle as a result of a low inflation state; and

FIG. 5 is a cutaway side view of a portion of a medical balloon showing helical fibers having a high low angle as a result of a higher inflation state.

The dimensions of some of the elements may be exaggerated relative to other elements for clarity or several physical components may be included in one functional block or element. Further, sometimes reference numerals may be repeated among the drawings to indicate corresponding or analogous elements. Moreover, some of the items depicted in the drawings may be combined into a single function.

### DETAILED DESCRIPTION

In the following detailed description, numerous specific details are set forth to provide a thorough understanding of the present invention. The disclosed embodiments may be practiced without these specific details. In other instances, well-known methods, procedures, components, or structures may not have been described in detail so as not to obscure the present invention.

The principles and operation of the apparatus and methods of the disclosure may be better understood with reference to the drawings and accompanying descriptions. The invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein are for the purpose of description and should not be regarded as limiting.

Certain features of the invention that are, for clarity, described in the context of separate embodiments, may also be provided in combination in a single embodiment. Conversely, various features of the invention that are, for brevity, described in the context of a single embodiment, may also be provided separately or in any suitable sub-combination.

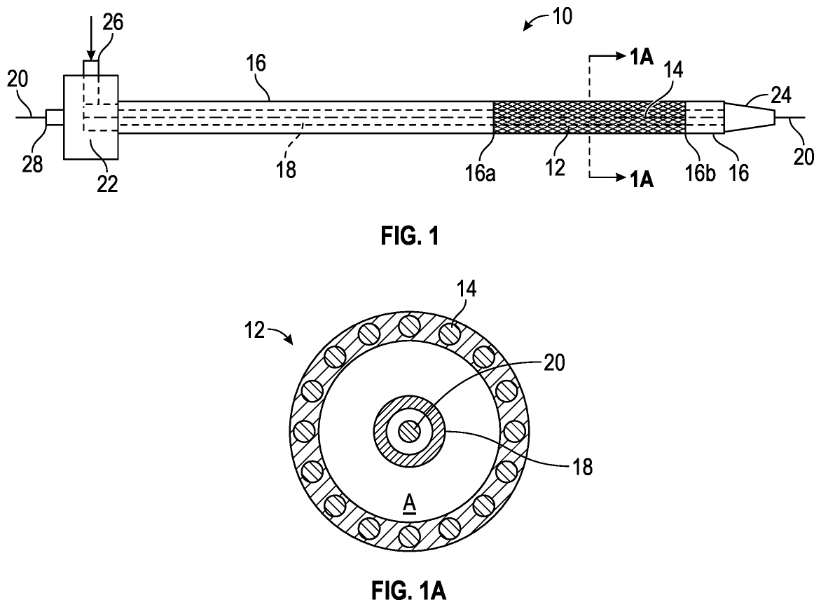

With reference to FIG. 1, a catheter 10 is shown, which includes an inflatable medical balloon 12. The medical balloon 12 comprises a compliant material including one or more embedded fibers 14 encapsulated within the compliant material. The compliant material may be an ultra-compliant material. For example, the ultra-compliant material may defined as any polymer with extensibility over 30% and Young's modulus less than 100 MPa. Examples of such a material include silicone, polyurethane, hydrogel, or combinations thereof. Such materials provide a high degree of compliance, puncture resistance, thin walls and, together with the use of embedded fibers, allow for high burst pressures to be achieved.

The one or more fibers 14 may be provided so as to form one or more fiber intersections. In one example, this may be achieved by providing the one or more fibers in a braided pattern, which may comprise a plurality of interwoven helical fibers, which thus form a plurality of such fiber intersections, each of which representing a crossing or braid angle. The braiding may be either at a consistent low winding angle or a variable winding angle along the length of the tubing. The one or more braided or intersecting fibers may have an axial crossing angle that may change as a result of radial expansion of the balloon 12, as outlined further in the following description.

The one or more fibers 14 may be formed of a variety of inelastic materials, including, but not limited to, Kevlar, Vectran, Spectra, Dacron, Dyneema, Turlon (PBT), Zylon (PBO), polyimide (PIM) and other ultrahigh molecular weight polyethylenes, aramids, and the like. In one embodiment, the fibers 14 may be aramid fibers, preferably multi-filament. In another embodiment, the fibers 14 may be para-aramid fibers, preferably multi-filament. In one example, the fibers 14 may be Technora brand paraphenylene/3,4-oxydiphenylene/terephthalamide copolymer, preferably multi-filament.

The catheter 10 including the balloon 12 may be constructed by attaching a tubing including the compliant material and embedded fibers to opposed ends 16a, 16b of a catheter shaft 16, which is dimensionally stable and thus non-compliant (that is, it does not radially expand upon the application of fluid pressure). The attachment may be achieved by bonding or welding the ends of the tubing to the corresponding ends 16a, 16b of the shaft 16. As can be understood from FIG. 1, the outer diameter of the tubing may be attached such that, in a nominal condition (that is, without the application of any inflation pressure), it lies substantially flush with the outer diameters of one or both of the adjacent shaft ends 16a, 16b. In this matter, the catheter 10 has a smooth, uninterrupted outer surface, which is thus well-adapted for passing through a vessel and, in particular, tortuous anatomy. This arrangement also avoids the need for pleating, wrapping or folding of the medical balloon prior to introduction into a vessel, which saves time and expense. The same benefit could also be obtained by attaching the ends of the tubing within the lumens of the shaft ends 16a, 16b, or in situations where such a smooth profile is not needed, attaching the tubing to the outer surfaces of the shaft ends.

As also shown in FIG. 1A, the catheter 10 may include an internal tube 18 within the shaft 16, which may form a lumen for a guidewire 20. This internal tube 18 may connect to a proximal hub 22 at a proximal end of the catheter 10, and may be sealed to a distal tip 24 at the distal end thereof, thereby fixing the length of the shaft 16 against longitudinal expansion. The hub 22 may include a first inflation port 26 for supplying inflation fluid from indeflator (not shown) to an annulus A between the outer surface of the inner tube 18 and the interior or inner surface of the balloon 12, and a second port 28 for receiving the guidewire 20, which may pass through the distal tip 24.

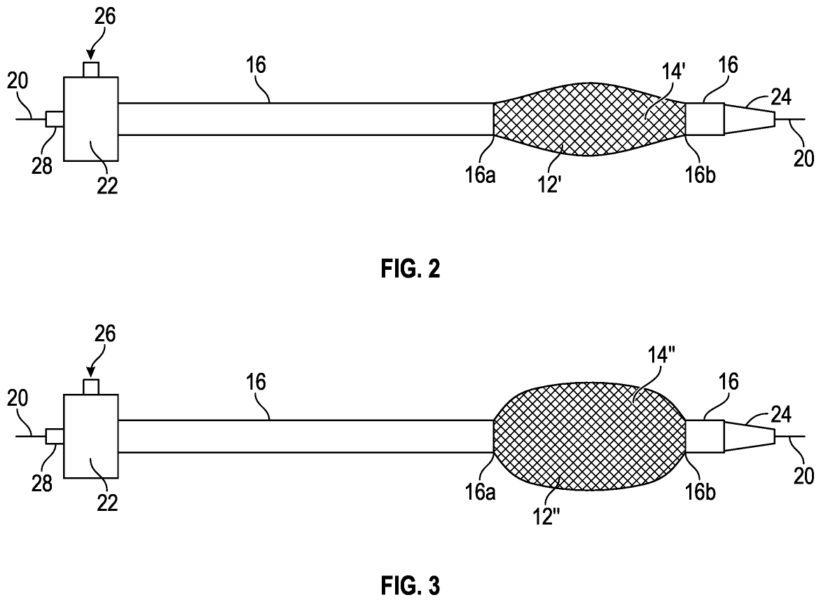

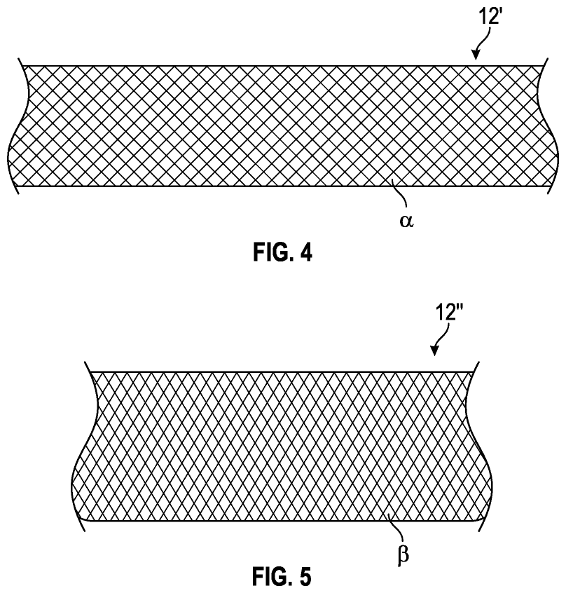

Thus, by applying inflation pressure to the shaft 16, and in particular the annulus A, via the inflation port 26, the medical balloon 12 may be caused to expand radially, such as to engage a lesion in a vessel. In other words, the medical balloon 12 together with the shaft 16 form a continuous interior compartment that may receive the inflation fluid, but only the tubing may expand to form the balloon 12 the result of compliance. The expansion may be variable, such as to a first inflated condition 12′ having a first diameter with the application of a first amount of fluid pressure, as shown in FIG. 2, and to a second inflated condition 12″ with a second, larger diameter upon the application of a second, increased amount of fluid pressure, as shown in FIG. 3. As can be appreciated, this may result in a change of the relative angles of the intersecting fiber(s) 14 from a first, lower angle α with an axial or longitudinal axis, as shown in FIG. 4 (fiber 14′), to a second, higher angle β with an axial or longitudinal axis, as shown in FIG. 5 (fiber 14″).

In connection with the use of a compliant material, this allows for the variable diameter of the balloon 12 to be achieved by corresponding increases or decreases in fluid pressure as applied to the shaft 16, which again is dimensionally stable and thus not amenable to change as a result of the application of fluid pressure. Consequently, the medical balloon 12 according to this disclosure is useful in treating vessels of a variety of diameters, as contrasted with non-compliant balloons that have a generally fixed nominal inflation diameter. Yet, the fibers 14 only allow for radial growth with increasing pressure in a controlled manner as they resist expansion of the compliant material in which they are embedded.

In order to form the compliant tubing, the one or more fibers 14 may be encased in a matrix material during a coextrusion process or by comolding. The one or more fibers 14 may also be applied over an outer surface of a compliant tubing, which may then be coated with additional compliant material to encapsulate the fibers therein. While a braided arrangement is shown, the fibers 14 may extend longitudinally, helically, or both, at any desired angle, and may be provided in one or more layers.

Summarizing, this disclosure may be considered to relate to the following items:
1. A medical apparatus, comprising:
   an inflatable medical balloon of a variable diameter comprising an ultra-compliant material having one or more fibers embedded therein.
2. The apparatus of item 1, wherein the ultra-compliant material is selected from the group comprising silicone, polyurethane, hydrogel, or any polymer with extensibility over 30% and Young's modulus less than 100 MPa.
3. The apparatus of item 1 or item 2, wherein the one or more fibers comprise one or more braided fibers.
4. The apparatus of any of items 1-3, wherein the one or more fibers comprise one or more fiber intersections having a first lower angle at a first lower state of inflation of the medical balloon and a second higher at a second higher state of inflation of the medical balloon.
5. The apparatus of any of items 1-4, further including a catheter shaft having opposed ends connected to a tubing forming the inflatable medical balloon.
6. The apparatus of item 5, wherein an outer diameter of the tubing is substantially flush with outer diameters of the opposed ends of the catheter shaft.
7. The apparatus of item 5 or item 6, further including a guidewire tube within the catheter shaft and the tubing forming the inflatable medical balloon, the guidewire tube connected to a distal tip of the catheter.
8. The apparatus of any of items 5-7, further including a hub at a proximal end of the catheter shaft including a first port for supplying an inflation fluid to the catheter shaft for expanding the tubing.
9. The apparatus of item 8, wherein the hub further includes a second port adapted for receiving a guidewire for passing through the catheter shaft.
10. A medical apparatus, comprising:
   a catheter shaft including opposed ends connected to a tubing formed of a compliant material with one or more fibers therein, the tubing forming an inflatable medical balloon of a variable diameter when the catheter shaft is pressurized as a result of the compliance of the compliant material.
11. The apparatus of item 10, wherein the compliant material comprises an ultra-compliant material.
12. The apparatus of item 11, wherein the ultra-compliant material is selected from the group comprising silicone, polyurethane, hydrogel, or any polymer with extensibility over 30% and Young's modulus less than 100 MPa.
13. The apparatus of any of items 10-12, wherein the one or more fibers comprise one or more braided fibers.
14. The apparatus of any of items 10-13, wherein the one or more fibers comprise one or more fiber intersections having a first lower angle at a first lower state of inflation of the medical balloon and a second higher at a second higher state of inflation of the medical balloon.
15. The apparatus of any of items 10-14, wherein an outer diameter of the tubing is substantially flush with outer diameters of the opposed ends of the catheter shaft.
16. The apparatus of any of items 10-15, further including a guidewire tube within the catheter shaft and the tubing forming the inflatable medical balloon, the guidewire tube connected to a distal tip of the catheter shaft.
17. The apparatus of any of items 10-16, further including a hub at a proximal end of the catheter shaft including a first port for supplying an inflation fluid to the catheter shaft for expanding the tubing.
18. The apparatus of item 17, wherein the hub further includes a second port adapted for receiving a guidewire for passing through the catheter shaft.
19. A medical apparatus, comprising:
   a catheter shaft connected to an inflatable medical balloon, the catheter shaft having an outer surface flush with an outer surface of the inflatable medical balloon.
20. The apparatus of item 19, wherein the medical balloon comprises a tubing formed of a compliant material with one or more fibers therein.
21. The apparatus of item 20, wherein the compliant material comprises an ultra-compliant material.
22. The apparatus of item 21, wherein the ultra-compliant material is selected from the group comprising silicone, polyurethane, hydrogel, or any polymer with extensibility over 30% and Young's modulus less than 100 MPa.
23. The apparatus of any of items 20-22, wherein the one or more fibers comprise one or more braided fibers.
24. The apparatus of any of items 20-23, wherein the one or more fibers comprise one or more fiber intersections having a first lower angle at a first lower state of inflation of the medical balloon and a second higher at a second higher state of inflation of the medical balloon.
25. The apparatus of any of items 19-24, wherein the medical balloon is attached to opposed ends of the catheter shaft.
26. The apparatus of any of items 19-25, further including a guidewire tube within the catheter shaft and the inflatable medical balloon, the guidewire tube connected to a distal tip of the catheter shaft.
27. The apparatus of any of items 19-26, further including a hub at a proximal end of the catheter shaft including a first port for supplying an inflation fluid to the catheter shaft for expanding the medical balloon.
28. The apparatus of item 27, wherein the hub further includes a second port adapted for receiving a guidewire for passing through the catheter shaft.

As used herein, the following terms have the following meanings:

“A”, “an”, and “the” as used herein refers to both singular and plural referents unless the context clearly dictates otherwise. By way of example, “a compartment” refers to one or more than one compartment.

“About,” “substantially,” or “approximately,” as used herein referring to a measurable value, such as a parameter, an amount, a temporal duration, and the like, is meant to encompass variations of +/−20% or less, including +/−10% or less, +/−5% or less, +/−1% or less, and +/−0.1% or less of and from the specified value, in so far such variations are appropriate to perform in the disclosed invention. However, it is to be understood that the value to which such modifiers refer is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

Although the invention has been described in conjunction with specific embodiments, many alternatives, modifications, and variations will be apparent to those skilled in the art. Accordingly, it embraces all such alternatives, modifications, and variations that fall within the spirit and scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, or identification of any reference in this application shall not be construed as an admission that such reference is available as prior art to the present disclosure.

1. A medical apparatus, comprising:
an inflatable medical balloon of a variable diameter comprising an ultra-compliant material having one or more fibers embedded therein.

2. The apparatus of claim 1, wherein the ultra-compliant material is selected from the group comprising silicone, polyurethane, hydrogel, or any polymer with extensibility over 30% and Young's modulus less than 100 MPa.

3. The apparatus of claim 1, wherein the one or more fibers comprise a braided pattern.

4. The apparatus of claim 1, wherein the one or more fibers comprise one or more fiber intersections having a first lower angle at a first lower state of inflation of the medical balloon and a second higher angle at a second higher state of inflation of the medical balloon.

5. The apparatus of claim 1, further including a catheter shaft having opposed ends connected to a tubing forming the inflatable medical balloon.

6. The apparatus of claim 5, wherein an outer diameter of the tubing is substantially flush with outer diameters of the opposed ends of the catheter shaft in an uninflated state.

7. The apparatus of claim 5, further including a guidewire tube within the catheter shaft and the tubing forming the inflatable medical balloon, the guidewire tube connected to a distal tip of the catheter.

8. The apparatus of claim 5, further including a hub at a proximal end of the catheter shaft including a first port for supplying an inflation fluid to the catheter shaft for expanding the tubing.

9. The apparatus of claim 8, wherein the hub further includes a second port adapted for receiving a guidewire for passing through the catheter shaft.

10. A medical apparatus, comprising:
a catheter shaft including opposed ends connected to a tubing formed of a compliant material with one or more fibers therein, the tubing configured to form an inflatable medical balloon of a variable diameter when the catheter shaft is pressurized as a result of the compliance of the compliant material.

11. The apparatus of claim 10, wherein the compliant material comprises an ultra-compliant material.

12. The apparatus of claim 11, wherein the ultra-compliant material is selected from the group comprising silicone, polyurethane, hydrogel, or any polymer with extensibility over 30% and Young's modulus less than 100 MPa.

13. The apparatus of claim 10, wherein the one or more fibers comprise one or more braided fibers.

14. The apparatus of claim 10, wherein the one or more fibers comprise one or more fiber intersections having a first lower angle at a first lower state of inflation of the medical balloon and a second higher angle at a second higher state of inflation of the medical balloon.

15. The apparatus of claim 10, wherein an outer diameter of the tubing is substantially flush with outer diameters of the opposed ends of the catheter shaft in an uninflated condition.

16. The apparatus of claim 10, further including a guidewire tube within the catheter shaft and the tubing forming the inflatable medical balloon, the guidewire tube connected to a distal tip of the catheter shaft.

17. The apparatus of claim 10, further including a hub at a proximal end of the catheter shaft including a first port for supplying an inflation fluid to the catheter shaft for expanding the tubing.

18. The apparatus of claim 17, wherein the hub further includes a second port adapted for receiving a guidewire for passing through the catheter shaft.

19. A medical apparatus, comprising:
a catheter shaft connected to an inflatable medical balloon, the catheter shaft having an outer surface flush with an outer surface of the inflatable medical balloon in an uninflated condition.

20. The apparatus of claim 19, wherein the medical balloon comprises a tubing formed of a compliant material with one or more fibers therein.

21-28. (canceled)
